(19) United States
(12) Patent Application Publication
(10) Pub. No.: US 2011/0123443 A1
(43) Pub. Date: May 26, 2011

(54) PANCREATIC BETA-CELL MASS BIOMARKER

(75) Inventors: Yun-Ping Zhou, East Brunswick, NJ (US); Andrew Howard, Park Ridge, NJ (US); Nancy Thornberry, Westfield, NJ (US)

(73) Assignee: MERCK & CO., INC., Rahway, NJ (US)

(21) Appl. No.: 12/936,827

(22) PCT Filed: Apr. 10, 2009
(86) PCT No.: PCT/US2009/040156

§ 371 Date: Oct. 7, 2010

Related U.S. Application Data
(60) Provisional application No. 61/124,941, filed on Apr. 21, 2008.

(51) Int. Cl. A61K 51/00; G01N 33/68; A61K 49/00; A61K 49/16; A61K 49/04

(52) U.S. Cl. 424/1.49; 435/7.25; 436/501; 424/9.1; 424/9.34; 424/9.4

(57) ABSTRACT

A biomarker for pancreatic beta-cell mass comprising measuring the levels of CFC1 in the serum of a subject is described. The biomarker provides a noninvasive means for measuring pancreatic beta cell mass that is particularly useful for monitoring the efficacy of treatments for metabolic disorders such as Type I or Type II diabetes, including pancreatic islet cell transplantations.

### BACKGROUND OF THE INVENTION

(1) Field of the Invention

The present invention relates to a biomarker for pancreatic beta-cell mass comprising measuring the levels of CFC1 in the serum of a subject. The biomarker and method provides a noninvasive means for measuring pancreatic beta cell mass that is particularly useful for monitoring the efficacy of treatments for metabolic disorders such as Type I or Type II diabetes.

(2) Description of Related Art

The reduction of pancreatic beta-cell mass (BCM) plays a critical role in the pathogenesis of both Type I and Type II diabetes. Autopsy data indicates that patients with type II diabetes often manifest over 50% reduction in BCM compared to non-diabetic individuals (Butler et al., Beta-cell deficit and increased beta-cell apoptosis in humans with type II diabetes, Diabetes, 52(1):102-110 (2003)). It is posited that the continued progressive loss of BCM is responsible for the deterioration of glycemic control and for the ultimate failure of several classes of oral hypoglycemia agents. Therefore, the art has recognized a need for non-invasive methods for measuring beta-cell mass.

Souza et al., in J. Clin. Invest. 116: 1506-1513 (2006) disclosed a positron emission tomography (PET) reported that that positron emission tomography (PET)-based quantitation of pancreatic radiolabeled VMAT2 receptors in diabetic rats is a non-invasive way to measure beta cell mass. WO2007005283 to Harris et al. disclosed non-invasive methods for determining the beta cell mass in the pancreas of a subject by administering to the subject an effective amount of a vesicular monoamine transporter type 2 (VMAT2)-specific radioligand; obtaining at least one computerized image of at least a portion of the pancreas of the subject; and quantitatively analyzing the computerized image in order to determine the beta cell mass in the pancreas of the subject. However, there remains a lack of reliable methods for measuring beta-cell mass changes. Measuring insulin or C-peptide or other blood biochemistry tests do not reliably measure BCM changes. There remains a need for noninvasive means for measuring pancreatic beta-cell mass. Such non-invasive assessment of BCM would provide an important tool for better understanding of the natural history of diabetes. Moreover, it would facilitate early diagnosis of diabetes and the monitoring the efficacy and durability of new therapeutic interventions, including pancreatic islet cell transplantions.

### SUMMARY OF THE INVENTION

It has recently been observed that CFC1 is highly and selectively expressed in primate pancreatic islet cells and that protein expression of CFC1 co-localizes with insulin-producing beta cells in the pancreas. Additionally, it has been discovered that CFC1 protein can be shed from cultured human islets in a glucose-independent manner. Therefore, CFC1 protein is a novel biomarker for the measurement of pancreatic islet beta-cell mass.

Therefore, the present invention provides a biomarker for monitoring pancreatic islet beta-cell mass comprising detecting and measuring the amount of CFC1 protein in the plasma, serum, or whole blood of a subject, particularly a subject that has II diabetes or type I diabetes following islet transplantation. The present invention provides a non-invasive method for measuring pancreatic islet beta-cell mass comprising the step of measuring circulation levels of CFC1 protein in plasma, serum, or whole blood obtained from a mammalian subject. Thus, there is provided a diagnostic tool for use in diabetic subjects comprising CFC1 protein and antibodies that are specific for the CFC1 protein. Such monitoring of CFC1 protein has potential as a diagnostic tool for BCM measurement, for example, in type 2 diabetes and in type 1 diabetes following islet transplantation.

There is further provided a method for monitoring the effects of agents on beta-cell mass comprising the steps of administering an agent and measuring the circulating levels of CFC1 protein in plasma or serum. Such agents may be selected from the group consisting of DPP4 inhibitors; GLP-1 receptor agonists; insulin-sensitizing agents; hepatic glucose production inhibitors; and glucagon receptor agonists or antagonists. This is particularly useful for monitoring the efficacy of a treatment regime for diabetes or the prognosis for islet transplantation procedure.

In addition to measuring the circulation levels of CFC1 in plasma, serum, or whole blood, monitoring CFC1 protein levels in pancreatic islet beta-cells in the subject can also be achieved by imaging means such as positron emission tomography (PET) or magnetic resonance imaging (MRI). These imaging modalities can be carried out using high affinity specific antibody or small molecules that specifically bind CFC1 protein and which are conjugated to a detection means, including but not limited to nanoparticles. The antibodies are preferably antibodies appropriate for the species of the subject. For example, for use in human subjects, the antibodies are preferably humanized antibodies. In addition, it is preferable that the antibodies be modified to have reduced or abrogated Fcγ receptor binding and to have reduced or abrogated inflammatory activity. The antibodies migrate to the pancreatic islet beta-cells and bind to the CFC1 protein on the surface of the beta-cells. The imaging means provides a view of the health of the subject's beta-cells as to whether the beta-cell mass is increasing or decreasing. The ability to monitor the increase or decrease of beta-cell mass is particularly useful for monitoring the success of pancreatic islet cell transplants and for the effect of various anti-diabetic agents on the health of the subject's beta-cell mass. Thus, anti-diabetic treatment regimes, including transplantation therapies, can be monitored for efficacy.

Therefore, the present invention provides an immunoassay method to measure pancreatic beta-cell mass in a subject using an antibody, which binds to CFC1 protein, the method comprising the steps of: (a) obtaining a biological sample from the subject; (b) contacting the biological sample with an antibody specific for CFC1 protein under conditions which allow binding of the CFC1 protein to the antibody; and (b) detecting the presence of the CFC1 protein in the biological sample, wherein the amount of the CFC1 protein detected in the sample provides a measurement of the pancreatic beta-cell mass in the subject.

In general, the biological sample is whole blood, serum, or plasma and the antibody, which can be a monoclonal or polyclonal antibody, can be in solution or bound to a solid phase support. In further aspects, the method further comprises the step of (c) comparing the amount of the CFC1 in the sample to a control value for the CFC1 protein.

In further still aspects, the biological samples are obtained from the subject over a period time and each sample is contacted with the antibody to detect the CFC1 protein.

Further provided is a method for monitoring the efficacy of a treatment regime for a metabolic disease in a subject comprising: (a) obtaining a first biological sample from the subject prior to the treatment regime and then after commencement of the treatment regime, obtaining one or more subsequent biological samples from the subject over time; (b) contacting each of the biological samples with an antibody specific for CFC1 protein; and (c) detecting the presence or lack thereof of the CFC1 protein in the biological samples, wherein detection and/or an increase in the amount of CFC1 protein in the biological samples over time indicates that the treatment regime is efficacious. The antibody can be a monoclonal antibody or a polyclonal antibody.

In particular aspects, the treatment regime comprises administering to the subject an agent selected from the group consisting of DPP4 inhibitors; GLP-1 receptor agonists; insulin-sensitizing agents; hepatic glucose production inhibitors; glucagon receptor agonists or antagonists, and combinations thereof.

In other aspects, the treatment regime comprises transplantation of pancreatic islet cells into the subject and the increase or maintenance of a detectable level of CFC1 protein over time indicates that the transplantation is efficacious.

Further provided is a method for measuring beta-cell mass in a subject comprising: (a) administering to the subject an antibody specific for detecting CFC1 protein conjugated to a detectable substance; and (b) monitoring the subject with a detection means to detect whether the antibody conjugate becomes associated with the beta-cells of the pancreas.

In particular aspects, the detection means is positron emission tomography (PET) or magnetic resonance imaging (MRI). In further aspects, the detectable substance is a short-lived radioisotope.

Further provided is a method for determining the beta cell mass in the pancreas of a subject comprising: (a) administering to the subject an effective amount of an antibody specific for CFC1 conjugated to a detectable substance (b) obtaining at least one computerized image of at least a portion of the pancreas of the subject; and (c) quantitatively analyzing the computerized image in order to determine the beta cell mass in the pancreas of the subject.

Further still, provided is a method for diagnosing a metabolic disorder in a subject comprising: (a) administering to the subject an effective amount of an antibody specific for CFC1 conjugated to a detectable substance; (b) obtaining at least one computerized image of at least a portion of the pancreas of the subject (c) quantitatively analyzing the computerized image in order to determine the beta cell mass in the pancreas of the subject; and (d) comparing the beta cell mass with a baseline measure of beta cell mass, where a decreased beta cell mass or increased beta cell mass versus the baseline measure is associated with the presence of a metabolic disorder

Further still, provided a method for assessing the prognosis of a subject at risk for developing diabetes comprising periodically: (a) administering to the subject an effective amount of an antibody specific for CFC1 conjugated to a detectable substance; (b) obtaining at least one computerized image of at least a portion of the pancreas of the subject (c) quantitatively analyzing the computerized image in order to determine the beta cell mass in the pancreas of the subject; and (d) comparing the periodically determined beta cell mass with a baseline measure of beta cell mass, where decreased beta cell mass versus the baseline measure is associated with the progression of preclinical diabetes to diabetes.

Further still is provided a method for determining the efficacy of a therapy for treating or preventing a metabolic disorder comprising periodically: (a) administering to the subject an effective amount of an antibody specific for CFC1 conjugated to a detectable substance; (b) obtaining at least one computerized image of at least a portion of the pancreas of the subject; (c) quantitatively analyzing the computerized image in order to determine the beta cell mass in the pancreas of the subject; and; (d) comparing the periodically determined beta cell mass with a baseline measure of beta cell mass, where a beta cell mass generally equivalent to the baseline measure, is indicative of a successful therapy to treatment or prevention of the metabolic disorder.

Further still, provided is a method for managing the treatment or prevention of diabetes comprising periodically: (a) administering to the subject an effective amount of an antibody specific for CFC1 conjugated to a detectable substance; (b) obtaining at least one computerized image of at least a portion of the pancreas of the subject; (c) quantitatively analyzing the computerized image in order to determine the beta cell mass in the pancreas of the subject; and (d) comparing the periodically determined beta cell mass with a baseline measure of beta cell mass, where a decreased beta cell mass versus the baseline measure is associated with the need for further therapy.

In particular aspects of the above imaging methods, the detectable substance is a radioligand and in further aspects, the computerized image is obtained using a positron emission tomography (PET).

In further still aspects, the antibody is a humanized monoclonal antibody and in further still aspects, the humanized monoclonal antibody has reduced or lacks inflammatory activity and effector function.

Also provided are the use of an antibody specific for CFC1 protein to measure pancreatic beta-cell mass and the use of CFC1 protein in the manufacture of a reagent for measuring pancreatic beta-cell mass.

### BRIEF DESCRIPTION OF THE DRAWINGS

FIG. 1 depicts expression of CFC1 in human islet cells. CFC1 is found to be specifically enriched in human islets as revealed by the Merck Body Atlas; the data is reflected as the ratio of islet intensity/reference-pool intensity. Two probes for CFC1 were used, highly islet-specific and highly expressed.

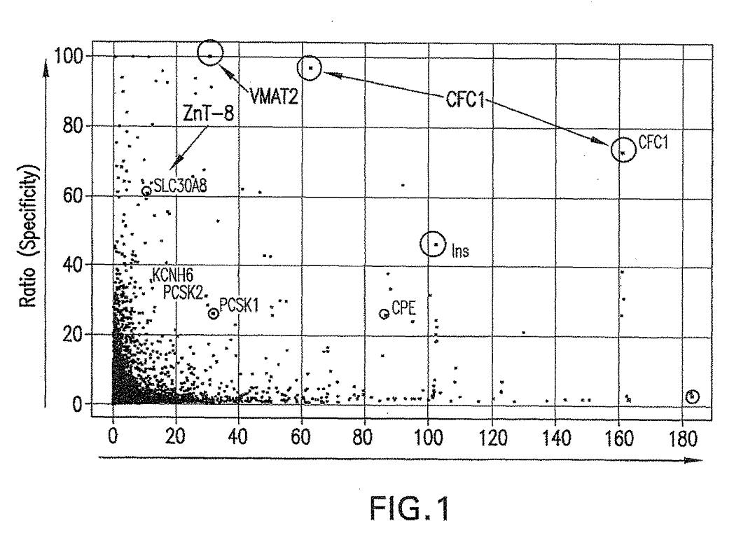

FIG. 2 shows TAQMAN real-time PCR confirmation of CFC1 mRNA expression in human islets. INS (insulin) was included as a reference. SLC80A8: zinc transporter ZnT-8. SLC18A2: vesicle monoamine transporter type 2 (VMAT2). Additional islet enriched biomarker targets measured in the same assay were: SLC7A1: cationic amino acid transporter, y+ system, member 1 (CAT-1). ABCC8: ATP-binding cassette, sub-family C, member 8; sulfonylurea receptor. HTR3A: 5-hydroxytryptamine (serotonin) receptor 3A.

FIG. 3 depicts the co-localization of CFC1 protein with insulin producing cells revealed by immunofluorescence staining in pancreatic sections from normal subjects.

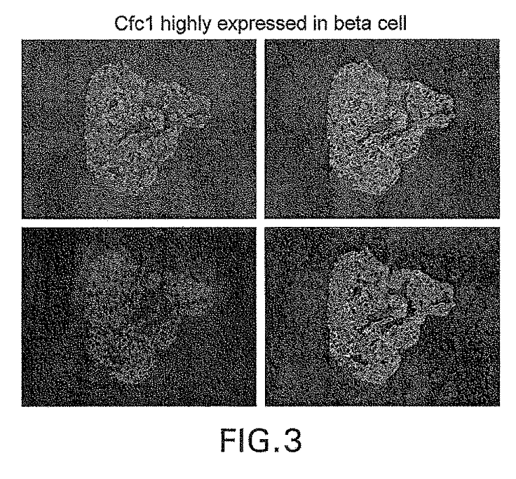

FIG. 4 demonstrates that CFC1 protein release in tissue culture is not affected by insulin and glucose levels. Human islets were cultured in RPMI 1640 medium with or without addition of phospholipase C (PLC). CFC1 protein accumulation in culture medium was measured by a sandwich ELISA.

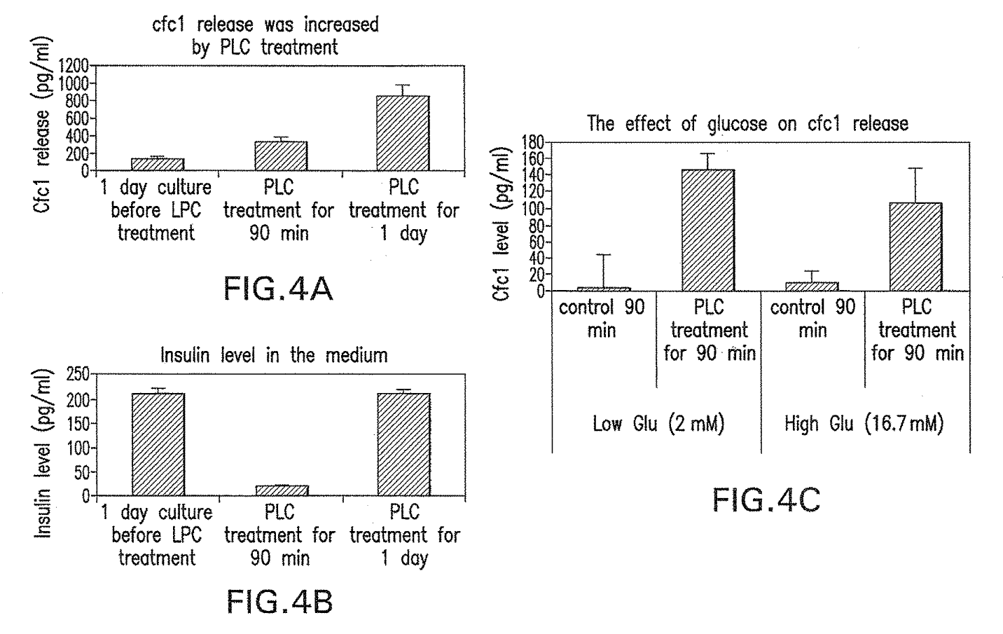

### DETAILED DESCRIPTION OF THE INVENTION

The present inventors have found that that CFC1 is expressed in primate pancreatic islet cells in a highly selective manner. It has been further observed that protein expression of CFC1 specifically co-localizes with insulin-producing beta cells in the pancreas. Additionally, CFC1 can be shed from cultured human islets in a glucose-independent manner. These observations suggested that measuring the circulating levels of CFC1 in a subject would provide an assessment of the state of the subject's pancreatic beta-cell health.

CFC1 encodes a member of the epidermal growth factor (EGF)-Cripto, Frl-1, and Cryptic (CFC) family (See Bamford et al., Loss-of-function mutations in the EGF-CFC gene CFC1 are associated with human left-right laterality defects, Nat. Genet. 26: 365-369 (2000). EGF-CFC family member proteins share a variant EGF-like motif, a conserved cysteine-rich domain, and a C-terminal hydrophobic region. These proteins play key roles in intercellular signaling pathways during vertebrate embryogenesis. Mutations in CFC1 can cause autosomal visceral heterotaxy. This protein is involved in left-right asymmetric morphogenesis during organ development. CFC1 is also known as FRL-1, cripto, cryptic, cryptic family 1, HTX2; CRYPTIC; FLJ77897; and MGC133213 (See also, Shen & Schier, Trends Genet. 16(7): 303-309 (2000)). CFC1 is a cell membrane glycoprotein attached by a cleavable glycosylphosphatidylinositol (GPI) anchor. The human CFC1 protein is encoded by SEQ ID NO:1 and has the amino acid sequence shown in SEQ ID NO:2 (See also GenBank Accession No. NP_{—}115934). CFC1 homologs include the rat CFC1 (GenBank Accession No. NP_{—}001102774) and the murine cripto (Genebank Accession No. BC100706.1). The mouse CFC1 protein is encoded by SEQ ID NO:3 and has the amino acid sequence shown in SEQ ID NO:4. The rat CFC1 protein is encoded by SEQ ID NO:5 and has the amino acid sequence shown in SEQ ID NO:6.

CFC1 is involved in the evolutionarily conserved establishment of left-right lateral asymmetry. Inactivation of Cfc1 in mice results in laterality defects and complex cardiac malformations. Similarly, mutations in the human CFC1 gene have been identified in patients with heterotaxy syndrome. The cardiac defects in humans resemble those in mice lacking CFC1. U.S. Pub. Application No. 2003/0207293 to Ducker discloses a cryptic-like protein with one amino acid difference from SEQ ID NO:2. U.S. Pat. No. 5,981,215 to Meissner et al. discloses a human criptin growth factor with an amino acid sequence partially similar to SEQ ID NO:2. Cripto-1, also referred to as cripto or CR-1, is not CFC1 but is a member of the epidermal growth factor (EGF)-CFC family of peptides. Cripto-1 has been used as a serologic marker in breast and colon cancer (Bianco et al., Identification of Cripto-1 as a novel serologic marker for breast and colon cancer, Clin. Cancer Res., 12(17):5158-64 (2006); U.S. Pat. No. 7,078,176 to Bianco et al.) and up-regulation in epithelial cancers (Hu & Ping, Cripto as a target for cancer immunotherapy, Expert Opin. Ther. Targets, 9(2): 383-94 (2005)). CFC1 protein has not previously been reported to be specifically enriched in pancreatic islet beta-cells and to be localized over the insulin-producing cells therein, so this recent unexpected finding suggests that CFC1 protein can serve as a biomarker for non-invasive means for measuring pancreatic islet beta-cell mass. Such a marker can be used as a diagnostic tool for monitoring beta-cell mass subjects that have a metabolic disease such as type I and type II diabetes, and type I diabetes that have received islet transplants.

Measuring CFC1 protein levels in a subject can be important for monitoring the efficacy of a treatment regime, including beta-cell or stem cell transplantation therapies, for a patient that has a metabolic disorder that is causing destruction or loss of pancreatic beta-cells, e.g., type I and type II diabetes. Thus, measuring the levels of CFC1 protein in the serum obtained from a subject over the time course of a treatment regime can provide a non-invasive means for determining the overall state of the subject's pancreatic beta-cell population and thus the effectiveness of the treatment regime or success of a transplantation therapy. Measuring CFC1 protein levels is a particularly useful diagnostic tool in the treatment and management of type I and type H diabetics. Additionally, CFC1 protein-based noninvasive measurement of beta-cell mass can be used to monitor in direct fashion the effects of various agents, including but not limited to DPP4 inhibitors; GLP-1 receptor agonists; insulin-sensitizing agents; hepatic glucose production inhibitors; and glucagon receptor agonists or antagonists.

Thus, in one aspect, provided is an immunoassay method to measure pancreatic beta-cell mass in a subject using an antibody, which binds to CFC1 protein, the method comprising the steps of (a) obtaining a biological sample from the subject; (b) contacting the biological sample with an antibody specific for CFC1 protein under conditions which allow binding of the CFC1 protein to the antibody; and (b) detecting the presence of the CFC1 protein in the biological sample, wherein the amount of the CFC1 protein in the sample provides a measurement of the pancreatic beta-cell mass in the subject. In general, the biological sample is whole blood, serum, or plasma. In further aspects, the method further comprises the step of (c) comparing the amount of the CFC1 in the biological sample to a control value for CFC1 protein in the subject. The term “control value” as used herein refers to a basal level of CFC1 that is present in the subject obtained prior to the commencement of a treatment regime or transplantation therapy. The present invention provides methods and compositions for determining control values for CFC1 protein. Such control values may need to account for age of the individual and therefore be directed to certain age ranges, as oxidative stress may accumulate over time. Such control values may additionally need to account for gender and race, and for environmental exposures, e.g., smoking, diet, etc.

In another aspect, Further provided is a method for monitoring the efficacy of a treatment regime for a metabolic disease in a subject comprising: (a) obtaining a first biological sample from the subject prior to the treatment regime and then after commencement of the treatment regime, obtaining one or more subsequent biological samples from the subject over time; (b) contacting each of the biological samples with an antibody specific for CFC1 protein; and (c) detecting the presence or lack thereof of the CFC1 protein in the biological samples, wherein detection and/or an increase in the amount of CFC1 protein in the biological samples over time indicates that the treatment regime is efficacious. The antibody can be a monoclonal antibody or a polyclonal antibody.

The term “detection” as used herein means determination that CFC1 protein is present. The methods and compositions of this invention can also be used to determine the amount of or concentration of CFC1 protein in a sample. Quantification and detection of CFC1 protein can be performed by any means known to those skilled in the art. Means of detection and quantification include but are not limited to precipitation of the CFC1 protein by an antibody which binds to the CFC1 protein, Western immunoblotting in which the CFC1 protein (either as part of a mixture or contained in an immunoprecipitated complex) is separated by gel electrophoresis, transferred to a suitable support (e.g., nitrocellulose) and visualized by reaction with an antibody(ies); radioimmunoassay, in which the degree to which the protein competes with a radioactively labeled standard for binding to the antibody is used as a means of detecting and quantifying the protein; and enzyme-linked immunosorbant assay (ELISA).

ELISA is a known technique for quantifying proteins in which, generally, an antibody against the protein of interest is immobilized on an inert solid, e.g., polystyrene. A sample to be assayed for the protein of interest is applied to the surface containing immobilized antibody. Protein binds the antibody, forming a complex. This complex is then contacted by a second antibody which binds the same protein and which is covalently bound to an easily assayed enzyme. After washing away any of the second antibody which is unbound, the enzyme in the immobilized complex is assayed, providing a measurement of the amount of protein in the sample. The ELISA procedure can be reversed, i.e., the antigen is immobilized on an inert support (e.g. 96-well microplate) and samples are probed for the presence of antibody to the immobilized antigen. The CFC1 protein can also be detected and its localization determined in cells and tissues using immunohistochemical procedures. For the present invention, ELISA, Western immunoblotting following electrophoretic separation of a protein mixture, and immunohistochemical procedures are useful methods of detecting and quantifying the CFC1 protein in a sample.

CFC1 protein can be detected and quantified in samples including, but not limited to, plasma and serum. These samples may be of human origin or they may be taken from animals other than humans, for example, rats, mice, monkeys, dogs, rabbits, and the like.

The present invention includes an immunoassay utilizing an antibody for CFC1 protein. The term “immunoassay” as used herein refers to a method of detecting or measuring antigens, in this case CFC1 protein, by using antibodies as reagents. The antibodies can be polyclonal or, preferably, monoclonal. The terms “polyclonal antibodies” and “monoclonal antibodies” have the standard meanings understood by those skilled in the art and refer to antibodies, either a mixture of different antibodies in the case of polyclonal antibodies, or a single antibody in the case of monoclonal antibodies, both of which are produced, in general, by immunization of an animal with an antigen. In the case of monoclonal antibodies, antibody-producing cells are selected from the animal and fused with myeloma cells. These cells are then cultured. The antibodies of the present invention detect CFC1 protein to a desired level.

Techniques for detecting antibody binding are well known in the art. Antibody binding to CFC1 may be detected through the use of chemical reagents that generate a detectable signal that corresponds to the level of antibody binding and, accordingly, to the level of CFC1 protein expression. In one of the immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system and Biocare Medical's Mach 3 system, may be used to practice the present invention.

In one particular immunocytochemistry method of the invention, antibody binding to CFC1 is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., CFC1 overexpression) and to determine beta-cell mass. Alternatively, samples may be reviewed via automated microscopy or by personnel with the assistance of computer software that facilitates the identification of positive staining cells.

The terms “antibody” and “antibodies” broadly encompass naturally occurring forms of antibodies and recombinant antibodies such as single-chain antibodies, chimeric and humanized antibodies and multi-specific antibodies as well as fragments and derivatives of all of the foregoing, which fragments and derivatives have at least an antigenic binding site. Antibody derivatives may comprise a protein or chemical moiety conjugated to the antibody.

“Antibodies” and “immunoglobulins” (Igs) are glycoproteins having the same structural characteristics. While antibodies exhibit binding specificity to an antigen, immunoglobulins include both antibodies and other antibody-like molecules that lack antigen specificity. Polypeptides of the latter kind are, for example, produced at low levels by the lymph system and at increased levels by myelomas.

The term “antibody” is used in the broadest sense and covers fully assembled antibodies, antibody fragments that can bind antigen (e.g., Fab′, F′(ab).sub.2, Fv, single chain antibodies, diabodies), and recombinant peptides comprising the foregoing.

The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al. (1995) Protein Eng. 8(10):1057 1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. In a two-chain Fv species, this region consists of a dimer of one heavy- and one light-chain variable domain in tight, non-covalent association. In a single-chain Fv species, one heavy- and one light-chain variable domain can be covalently linked by flexible peptide linker such that the light and heavy chains can associate in a “dimeric” structure analogous to that in a two-chain Fv species. It is in this configuration that the three CDRs of each variable domain interact to define an antigen-binding site on the surface of the V_{H} V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy-chain C_{H1} domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them.

Polyclonal antibodies can be prepared by immunizing a suitable subject (e.g., rabbit, goat, mouse, or other mammal) with a biomarker protein immunogen. The antibody titer in the immunized subject can be monitored over time by standard techniques, such as with an enzyme linked immunosorbent assay (ELISA) using immobilized biomarker protein. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495 497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77 96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:550 52; Kenneth (1980) in Monoclonal Antibodies: A New Dimension In Biological Analyses (Plenum Publishing Corp., NY); and Lemer (1981) Yale J. Biol. Med., 54:387 402).

Alternative to preparing monoclonal antibody-secreting hybridomas, a monoclonal antibody can be identified and isolated by screening a recombinant combinatorial immunoglobulin library (e.g., an antibody phage display library) with CFC1 to thereby isolate immunoglobulin library members that bind the biomarker protein. Kits for generating and screening phage display libraries are commercially available (e.g., the Pharmacia Recombinant Phage Antibody System, Catalog No. 27-9400-01; and the Stratagene SURFZAP Phage Display Kit, Catalog No. 240612). Additionally, examples of methods and reagents particularly amenable for use in generating and screening antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370 1372; Hay et al. (1992) Hum. Antibod. Hybridomas 3:81 85; Huse et al. (1989) Science 246:1275 1281; Griffiths et al. (1993) EMBO J. 12:725 734.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, diehlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art, video-microscopy and software methods for the quantitative determination of an amount of multiple molecular species (e.g., CFC1 protein) in a biological sample wherein each molecular species present is indicated by a representative dye marker having a specific color. Such methods are also known in the art as a calorimetric analysis methods. In these methods, video-microscopy is used to provide an image of the biological sample after it has been stained to visually indicate the presence of CFC1 protein. Some of these methods, such as those disclosed in U.S. Ser. No. 09/957,446 to Marcelpoil et al. and U.S. Ser. No. 10/057,729 to Marcelpoil et al., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for the CFC1 protein. Methods for making antibodies and for selecting appropriate antibodies are known in the art. See, for example, Celis, ed. (in press) Cell Biology & Laboratory Handbook, 3rd edition (Academic Press, New York), which is herein incorporated in its entirety by reference. In some embodiments, commercial antibodies directed to CFC1 protein may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

In addition to measuring the circulation levels of CFC1 in plasma, serum, or whole blood, CFC1 protein can be measured in pancreatic islet beta-cells in the subject using an imaging or detection means such as positron emission tomography (PET) or magnetic resonance imaging (MRI). These imaging or detection modalities can be carried out using high affinity specific antibody or small molecules that specifically bind CFC1 protein. In general, antibodies specific for CFC1 protein are conjugated to a detectable substance, including but not limited to short-lived radioisotopes and nanoparticles, which are then administered to the subject intravenously. Examples of short-lived radioligands include but are not limited to ⁶⁴Cu, ⁷⁶Br, ¹²⁴I, ¹¹C, ¹³N, ¹⁵O, and ¹⁸F (See Voss et al., Positron emission tomography (PET) imaging of neuroblastoma and melanoma with ⁶⁴Cu-SarAr immunoconjugates, Proc. Natl. Acad. Sci. USA 104: 17489-17493 (2007)) for a discussion of radioligands that can be conjugated to antibodies for PET. The antibodies are preferably antibodies appropriate for the species of the subject. For example, for use in human subjects, the antibodies are preferably humanized antibodies. In addition, it is preferable that the antibodies be modified to have reduced or abrogated Fcγ receptor binding (lacks effector function) and to have reduced or abrogated (lack) inflammatory activity. For example, antibodies having sialylated N-glycans have reduced inflammatory activity (See Kaneko et al., Science 313(5787): 670-3 (2006); Nimmerjahn & Ravetch, J. Exper. Med., 204: 11-15 (2007); Nimmerjahn et al., Science 320(5874): 373-6 (2008)). The antibodies migrate to the pancreatic islet beta-cells and bind to the CFC1 protein on the surface of the beta-cells. The imaging means provides a view of the health of the subject's beta-cells as to whether the beta-cell mass is increasing or decreasing. The ability to monitor the increase or decrease of beta-cell mass is particularly useful for monitoring the success of pancreatic islet cell transplants and for the effect of various anti-diabetic agents on the health of the subject's beta-cell mass. Thus, anti-diabetic treatment regimes, including transplantation therapies, can be monitored for efficacy.

Therefore, in light of the foregoing, further provided is a method for determining the beta cell mass in the pancreas of a subject comprising: (a) administering to the subject an effective amount of an antibody specific for CFC1 conjugated to a detectable substance (b) obtaining at least one computerized image of at least a portion of the pancreas of the subject; and (c) quantitatively analyzing the computerized image in order to determine the beta cell mass in the pancreas of the subject. Further provided is a method for diagnosing a metabolic disorder in a subject comprising: (a) administering to the subject an effective amount of an antibody specific for CFC1 conjugated to a detectable substance; (b) obtaining at least one computerized image of at least a portion of the pancreas of the subject (c) quantitatively analyzing the computerized image in order to determine the beta cell mass in the pancreas of the subject; and (d) comparing the beta cell mass with a baseline measure of beta cell mass, where a decreased beta cell mass or increased beta cell mass versus the baseline measure is associated with the presence of a metabolic disorder

Further still is provided a method for assessing the prognosis of a subject at risk for developing diabetes comprising periodically: (a) administering to the subject an effective amount of an antibody specific for CFC1 conjugated to a detectable substance; (b) obtaining at least one computerized image of at least a portion of the pancreas of the subject (c) quantitatively analyzing the computerized image in order to determine the beta cell mass in the pancreas of the subject; and (d) comparing the periodically determined beta cell mass with a baseline measure of beta cell mass, where decreased beta cell mass versus the baseline measure is associated with the progression of preclinical diabetes to diabetes.

Further still is provided a method for determining the efficacy of a therapy for treating or preventing a metabolic disorder comprising periodically: (a) administering to the subject an effective amount of an antibody specific for CFC1 conjugated to a detectable substance; (b) obtaining at least one computerized image of at least a portion of the pancreas of the subject; (c) quantitatively analyzing the computerized image in order to determine the beta cell mass in the pancreas of the subject; and; (d) comparing the periodically determined beta cell mass with a baseline measure of beta cell mass, where a beta cell mass generally equivalent to the baseline measure, is indicative of a successful therapy to treatment or prevention of the metabolic disorder.

Further still, provided is a method for managing the treatment or prevention of diabetes comprising periodically: (a) administering to the subject an effective amount of an antibody specific for CFC1 conjugated to a detectable substance; (b) obtaining at least one computerized image of at least a portion of the pancreas of the subject; (c) quantitatively analyzing the computerized image in order to determine the beta cell mass in the pancreas of the subject; and (d) comparing the periodically determined beta cell mass with a baseline measure of beta cell mass, where a decreased beta cell mass versus the baseline measure is associated with the need for further therapy.

In particular aspects of the above imaging methods, the detectable substance is a radioligand and in further aspects, the computerized image is obtained using a positron emission tomography (PET).

Further provided is a kit for measuring beta-cell mass comprising a vial containing antibodies for detecting CFC1 protein and optionally a second antibody conjugated to a detectable substance. The kit further includes instructions for its use.

Examples are provided below further illustrating different features of the present invention and illustrate useful embodiments for practicing the invention. Theses embodiments should be viewed as exemplary of the present invention rather than in any way limiting its scope.

### Example 1

This Example shows that CFC1 is specifically enriched in human islets.

The custom ink-jet microarrays used for the Merck Monkey Body Atlas were manufactured by Agilent Technologies (Palo Alto, Calif.) and consisted of 47272 oligonucleotides extracted from human Unigene clusters and combined with RefSeq sequences and RIKEN full-length cDNA clones (due to the unavailability of finished monkey whole genome sequence). Sixty-six different primate tissues, including pancreatic islets (Rhesus monkey and human islet), were included in the Monkey Body Atlas. Total RNA from all tissues was extracted using Trizol reagent (Invitrogen, CA), reverse transcribed, and labeled with either Cy3 or Cy5 fluorochrome.

For a given sample, labeled complementary RNA (cRNA) was hybridized against a pool of 10 tissue cRNA as the reference. Gene expression measures are reported as (1) the hybridization intensity values, which reflects gene expression abundance, and (2) the ratio of the intensity values of islets over that of the reference pool, which correlates with tissue expression specificity. Rank ordering of genes by both measures led to the identification of highly islet-specific and enriched genes. This was followed by a bioinformatics analysis to select the genes whose protein products localized extracellularly or to cell surface. Unexpectedly, the secreted protein CFC1, represented by two independent probes (Reporter ID 10023834931 and 10023817081), was identified as a highly abundant and specific protein in human and monkey islets.

### Example 2

TAQMAN real-time RT-PCR of RNA from human pancreatic islets confirms that CFC1 expressed by the human islet cells.

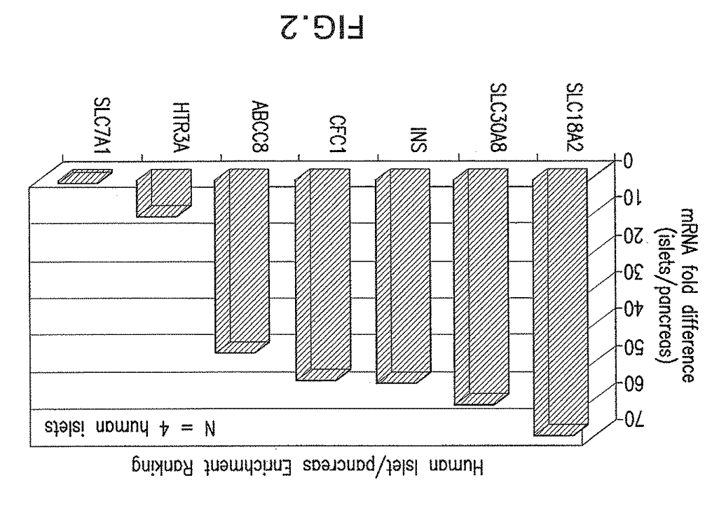

The TAQMAN assay was performed using the specific primer and probe set designed for the human CFC1 gene from ABI (Cat# Hs00414425_ml). The relative mRNA levels were normalized to beta-actin and the data was calculated based on the average of human islets from four donors. The ratio of mRNA in islets over whole pancreas was calculated and ranked from the highest to lowest. The results are shown in FIG. 2.

### Example 3

In this example, immunofluorescence staining of beta-cells showed that CFC1 co-localized with cells that produce insulin.

Paraffin sections of normal human pancreas were de-waxed and rehydrated, followed by three washes with PBS. After a one hour blocking step to remove non-specific antigens by incubating with 5% donkey serum, pancreas sections were incubated with anti-human CFC1 sheep serum (CFC1 antibody, R&D systems) and anti-guinea pig insulin serum (insulin antibody, Sigma) overnight at 4° C. After extensive washes with PBS, pancreas sections were cultured with Fluorescein-conjugated donkey anti-sheep secondary antibody and rhodamine-conjugated donkey anti-guinea pig secondary antibody (Jackson ImmunoResearch Lab) for 30 minutes at room temperature. Stained sections were mounted in Vectashield mounting medium with DAPI and analysed with a fluorescence microscope. hCFC1 was found to be highly expressed in pancreatic islets and co-localized with insulin-producing beta cells.

### Example 4

This Example shows that CFC1 release is not affected by insulin and glucose levels.

Equal numbers of human islets from normal subjects were cultured in 2 mL of RPMI-1640 medium supplemented with 10% serum, 1% ampicillin-strep in a six-well plate. Islets were cultured without or with 0.5 μg/mL phosphatidylinositol-specific phospholipase C (PI-PLC) for certain times as indicated. After incubation, culture medium was collected and accumulated CFC1 and insulin content was measured by an in house-developed CFC1 ELISA sandwich assay and insulin ELISA kit (ALPCO, Diagnostics). CFC1 release from human islets was increased by PI-PLC treatment, however the release of CFC1 was not correlated with insulin levels. To test the effect of glucose on CFC1 release, human islets treated without or with PI-PLC were cultured with low glucose (2 mM) or high glucose (16.7 mM) for 90 minutes. Released CFC1 level from human islets treated with high glucose was similar as that from human islets treated with low glucose.

### Sequences

TABLE 1

| SEQ | | |
|---|---|---|
| ID | | |
| NO: | Description | Sequence |
| 1 | Human | CACTGCGCCCCCGACCCTAGCTCAGGGACTGCAGAACTCA |
| | CFC1 | AGATACCATCCCGTTTCTCCTGGCTGAGGAAGGGAAGGGA |
| | (NM_032545) | ACATCCACATCTTCTGTACTCGTCCATTCTGTGTCCCCGGG |
| | Codons | GCCTGGAGTAAAGACACCTTCAAATGCAGAGACTCTTCAG |
| | 223-894 | ATTCAGCTTTCCTGGAAACTGATCTTCAATGCACTAAGAGA |
| | | AGGAGACTCTCAAACCAAAAATGACCTGGAGGCACCATGT |
| | | CAGGCTTCTGTTTACGGTCAGTTTGGCATTACAGATCATCA |
| | | ATTTGGGAAACAGCTATCAAAGAGAGAAACATAACGGCGG |
| | | TAGAGAGGAAGTCACCAAGGTTGCCACTCAGAAGCACCGA |
| | | CAGTCACCGCTCAACTGGACCTCCAGTCATTTCGGAGAGGT |
| | | GACTGGGAGCGCCGAGGGCTGGGGGCCGGAGGAGCCGCTC |
| | | CCCTACTCCCGGGCTTTCGGAGAGGGTGCGTCCGCGCGGCC |
| | | GCGCTGCTGCAGGAACGGCGGTACCTGCGTGCTGGGCAGC |
| | | TTCTGCGTGTGCCCGGCCCACTTCACCGGCCGCTACTGCGA |
| | | GCATGACCAGAGGCGCAGTGAATGCGGCGCCCTGGAGCAC |
| | | GGAGCCTGGACCCTCCGCGCCTGCCACCTCTGCAGGTGCAT |
| | | CTTCGGGGCCCTGCACTGCCTCCCCCTCCAGACGCCTGACC |
| | | GCTGTGACCCGAAAGACTTCCTGGCCTCCCACGCTCACGGG |
| | | CCGAGCGCCGGGGGCGCGCCCAGCCTGCTACTCTTGCTGCC |
| | | CTGCGCACTCCTGCACCGCCTCCTGCGCCCGGATGCGCCCG |
| | | CGCACCCTCGGTCCCTGGTCCCTTCCGTCCTCCAGCGGGAG |
| | | CGGCGCCCCTGCGGAAGGCCGGGACTTGGGCATCGCCTTT |
| | | AATTTTCTATGTTGTAAATAATAGATGTGTTTAGTTTACCGT |
| | | AAGCTGAAGCACTGGGTGAATATTTTTATTGGGTAATAAAT |
| | | ATTTTCATGAAAGCGCCTTTGGCTCCAGATCCTT |
| 2 | Human | MTWRHHVRLLFTVSLALQIINLGNSYQREKHNGGREEVTKVA |
| | CFC1 | TQKHRQSPLNWTSSHFGEVTGSAEGWGPEEPLPYSRAFGEGA |
| | protein | SARPRCCRNGGTCVLGSFCVCPAHFTGRYCEHDQRRSECGAL |
| | | EHGAWTLRACHLCRCIFGALHCLPLQTPDRCDPKDFLASHAH |
| | | GPSAGGAPSLLLLLPCALLHRLLRPDAPAHPRSLVPSVLQRERR |
| | | PCGRPGLGHRL |
| 3 | Mouse | CAGGACTGTATAGGGTCAGCACTTCCAGCCTGGTGGTTCAG |
| | CFC1 | AGCTCCTGACCTGAGAGGGCTTCAACACCTGGACTCCAGG |
| | (NM_007685) | ATCTTCCTTTAACCCTGCTGTCTCTGGTCCAGGCAGAGGGC |
| | codons | AGAGACATCTTCATCTTGCAAGACTGTGCATCCTGTAACCT |
| | 256-864 | GCTATAGTGATTCCAAGACCTGGAGTAAAGGGTGCCTCCG |
| | | GGGCTAGGATATTTGAGTTTCAACTTCTGTGGTCATCGATC |
| | | CCCAAGCACAGATGAGAGCGAACTCACCAACCCAGGGTAT |
| | | CAGTTTGAAAATGCATCAAGCCAGGCCTCTGTTTTTGGTGA |
| | | CTGTCGCGCTGCAGCTCATCGGTCTGGGATACAGTTATCAG |
| | | AGCGAAGGAGATGGTGCCAGAGAAGTCAGCAATATCCTCT |
| | | CTCCAGTGATCCCCGGGACGACACTGGACAGAACTCTGAG |
| | | TAATTCCAGCAGAAAGAATGACATTCCGGAGGGGGCGCGC |
| | | CTATGGGATTCCCTTCCTGACTCCAGCACTTTGGGAGAGAG |
| | | TGCAGTCCCTGTATCCCGCTGTTGCCACAATGGCGGCACCT |
| | | GTGTTCTGGGCAGTTTCTGCGTGTGCCCTGCCTATTTCACTG |
| | | GTCGCTATTGCGAGCACGACCAGAGGCGCAGAGACTGTGG |
| | | TGCCCTAGGGCATGGAGCTTGGACCCTGCACAGCTGCCGCC |
| | | TATGCAGGTGCATCTTCTCAGCCCTGTACTGCCTCCCACAC |
| | | CAAACGTTCAGCCACTGTGACCTGAAAAGCTTCCTTTCTTC |
| | | AGGCGCCAGAGGATCAAGAGAATGCAGCATCCCAAGCCTC |
| | | CTCCTGCTGGTGCTCTGCCTCCTCCTGCAGGGTGTGGCTGG |
| | | TAAGGGCTGAGGCTCCTAAGTGCGATGATAGACTCTCCTCC |
| | | TGAGCTGTCACCCTTGATTACACCACAGTGTGCCAGCAAGA |
| | | AAGCTGGGTGGTGGGCATCTGACTTGGTGTTGTGTCCTGTA |
| | | AATAACAGATTCACTGGAATATGCTGGATTCTCATGCTGTA |
| | | CAATAAAGAGGCTTAATGGT |
| 4 | Mouse | MRANSPTQGISLKMHQARPLFLVTVALQLIGLGYSYQSEGDG |
| | CFC1 | AREVSNILSPVIPGTTLDRTLSNSSRKNDIPEGARLWDSLPDSST |
| | protein | LGESAVPVSRCCHNGGTCVLGSFCVCPAYFTGRYCEHDQRRR |
| | | DCGALGHGAWTLHSCRLCRCIFSALYCLPHQTFSHCDLKSFLS |
| | | SGARGSRECSIPSLLLLVLCLLLQGVAGKG |
| 5 | Rat CFC1 | ATGCAGATCCCGAAGGCTAGCTATGCGCGCATCCGGAACC |
| | (XM_576538) | CTAGCCCAGGAAGACCTTGGCTAACGTCCCCAGTGATGAC |
| | Codons | GTATGTGAAAAAGCAAACCCTATGGGCACGTTGCCCGTCC |
| | 1-675 | ACCCAGAGTCTCTTCACATCAGCTGCACAGCTGCTTATCCA |
| | | TTTGTGGAGCGGGCAGTTGAGAGAAGGAGATGGTGCCAGA |
| | | GAAATTAGCTATCTCCTGTCTCCAAAGCTCCCAGGGACGAC |
| | | ACTGGATGGAACCCTGAGTAGTTCCAGTAGAAAGAATGAC |
| | | AGCCAGGAGGGAGCGCACCCGTCGGAGGCCCTTTCTGGTT |
| | | CCAACACTTTGGAGGAGAGTGCGGTCCCTGGATCCCGCTGT |
| | | TGCCACAATGGAGGCACCTGCGTTCTGGGCAGTTTCTGCGT |
| | | GTGCCCCGCGCACTTCACAGGTCGCTACTGCGAGCACGACC |
| | | AGAGACACAGAGACTGTGGCGCACTGGGGCACGGAGCTTG |
| | | GACCCTGCACAGCTGCCGCCTATGCAGGTGCATCTTCTCAG |
| | | CCCTTTACTGCCTCCCACGCCAAACATTCAGCCACTGTGAC |
| | | CTGAAAAGCTTCCTCTCTTCAGGCGCCAGAGGATCAAGGG |
| | | CATGCAGCATCCCGAGACTCCTCCTGCTGGTGCTCTGCCTC |
| | | CTCCTGCAGGTTGTGTGTGGCTGGTAAGGGCTGAGGCTCTG |
| | | AACTGCCCTGACAGAATCTCCTCCTGAGCCTGCAAGGTGTC |
| | | ACCCTTGACTACCCATGGCACCACAGCACGCCTGCAGGAA |
| | | AGCTGGGTGGTGGGTATCTGCTTCAGTGTTGAATGCTGTAA |
| | | ATGACCGAATGGCTGAAATATGCTGGATCCTCGTGCTGTGC |
| | | AATAAAGACGTTCAACAGCGA |
| 6 | Rat CFC1 | MQIPKASYARIRNPSPGRPWLTSPVMTYVKKQTLWARCPSTQ |
| | protein | SLFTSAAQLLIHLWSGQLREGDGAREISYLLSPKLPGTTLDGTL |
| | | SSSSRKNDSQEGAHPSEALSGSNTLEESAVPGSRCCHNGGTCV |
| | | LGSFCVCPAHFTGRYCEHDQRHRDCGALGIIGAWTLHSCRLC |
| | | RCIFSALYCLPRQTFSHCDLKSFLSSGARGSRACSIPRLLLLVLC |
| | | LLLQVVCGW |

Other embodiments are within the scope of the following claims. All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. All such variations apparent to those skilled in the art are deemed to be within the spirit, scope and concept of the invention as defined by the appended claims.

1. An immunoassay method to measure pancreatic beta-cell mass in a subject using an antibody, which binds to CFC1 protein, the method comprising the steps of:
(a) obtaining a biological sample from the subject;
(b) contacting the biological sample with an antibody specific for CFC1 protein under conditions which allow binding of the CFC1 protein to the antibody; and
(c) detecting the presence of the CFC1 protein in the biological sample, wherein the amount of the CFC1 protein detected in the sample provides a measurement of the pancreatic beta-cell mass in the subject.

2. The method of claim 1, wherein the biological sample is whole blood, serum, or plasma.

3. The method of claim 1, wherein the antibody is bound to a solid phase support.

4. The method of claim 1 further comprising the step of:
(d) comparing the amount of the CFC1 in the sample to a control value for the CFC1 protein.

5. The method of claim 1, wherein biological samples are obtained from the subject over a period time and each sample is contacted with the antibody to detect the CFC1 protein.

6. The method of claim 1, wherein the antibody is a monoclonal antibody.

7-10. (canceled)

11. A method for measuring beta-cell mass in a subject comprising:
(a) administering to the subject an antibody specific for detecting CFC1 protein conjugated to a detectable substance; and
(b) monitoring the subject with a detection means to detect whether the antibody conjugate becomes associated with the beta-cells of the pancreas.

12. The method of claim 11, wherein the detectable substance is a short-lived radioisotope.

13. The method of claim 11, wherein the detection means is positron emission tomography (PET) or magnetic resonance imaging (MRI).

14. The method of claim 11, wherein the antibody is a humanized monoclonal antibody.

15. The method of claim 11, wherein the humanized monoclonal antibody has reduced or lacks inflammatory activity and effector function.

16. A method for determining the beta cell mass in the pancreas of a subject comprising:
(a) administering to the subject an effective amount of an antibody specific for CFC1 conjugated to a detectable substance;
(b) obtaining at least one computerized image of at least a portion of the pancreas of the subject; and
(c) quantitatively analyzing the computerized image in order to determine the beta cell mass in the pancreas of the subject.

17. The method of claim 16, wherein the detectable substance is a short-lived radioisotope.

18. The method of claim 16, wherein the detection means is positron emission tomography (PET) or magnetic resonance imaging (MRI).

19. The method of claim 16, wherein the antibody is a humanized monoclonal antibody.

20. The method of claim 16, wherein the humanized monoclonal antibody has reduced or lacks inflammatory activity and effector function.

21-22. (canceled)


SEQUENCE LISTING (6 sequences)

SEQ ID NO: 1 - DNA, 1009 bp, Homo sapiens
  CDS (223)...(894): Encodes CFC1
cactgcgcccccgaccctagctcagggactgcagaactcaagataccatcccgtttctcctggctgaggaagggaagggaacatccacatcttctgtactcgtccattctgtgtccccggggcctggagtaaagacaccttcaaatgcagagactcttcagattcagctttcctggaaactgatcttcaatgcactaagagaaggagactctcaaaccaaaaatgacctggaggcaccatgtcaggcttctgtttacggtcagtttggcattacagatcatcaatttgggaaacagctatcaaagagagaaacataacggcggtagagaggaagtcaccaaggttgccactcagaagcaccgacagtcaccgctcaactggacctccagtcatttcggagaggtgactgggagcgccgagggctgggggccggaggagccgctcccctactcccgggctttcggagagggtgcgtccgcgcggccgcgctgctgcaggaacggcggtacctgcgtgctgggcagcttctgcgtgtgcccggcccacttcaccggccgctactgcgagcatgaccagaggcgcagtgaatgcggcgccctggagcacggagcctggaccctccgcgcctgccacctctgcaggtgcatcttcggggccctgcactgcctccccctccagacgcctgaccgctgtgacccgaaagacttcctggcctcccacgctcacgggccgagcgccgggggcgcgcccagcctgctactcttgctgccctgcgcactcctgcaccgcctcctgcgcccggatgcgcccgcgcaccctcggtccctggtcccttccgtcctccagcgggagcggcgcccctgcggaaggccgggacttgggcatcgcctttaattttctatgttgtaaataatagatgtgtttagtttaccgtaagctgaagcactgggtgaatatttttattgggtaataaatattttcatgaaagcgcctttggctccagatcctt

SEQ ID NO: 2 - protein, 223 aa, Homo sapiens
MTWRHHVRLLFTVSLALQIINLGNSYQREKHNGGREEVTKVATQKHRQSPLNWTSSHFGEVTGSAEGWGPEEPLPYSRAFGEGASARPRCCRNGGTCVLGSFCVCPAHFTGRYCEHDQRRSECGALEHGAWTLRACHLCRCIFGALHCLPLQTPDRCDPKDFLASHAHGPSAGGAPSLLLLLPCALLHRLLRPDAPAHPRSLVPSVLQRERRPCGRPGLGHRL

SEQ ID NO: 3 - DNA, 1038 bp, Mus musculus
  CDS (256)...(864): Encodes CFC1
caggactgtatagggtcagcacttccagcctggtggttcagagctcctgacctgagagggcttcaacacctggactccaggatcttcctttaaccctgctgtctctggtccaggcagagggcagagacatcttcatcttgcaagactgtgcatcctgtaacctgctatagtgattccaagacctggagtaaagggtgcctccggggctaggatatttgagtttcaacttctgtggtcatcgatccccaagcacagatgagagcgaactcaccaacccagggtatcagtttgaaaatgcatcaagccaggcctctgtttttggtgactgtcgcgctgcagctcatcggtctgggatacagttatcagagcgaaggagatggtgccagagaagtcagcaatatcctctctccagtgatccccgggacgacactggacagaactctgagtaattccagcagaaagaatgacattccggagggggcgcgcctatgggattcccttcctgactccagcactttgggagagagtgcagtccctgtatcccgctgttgccacaatggcggcacctgtgttctgggcagtttctgcgtgtgccctgcctatttcactggtcgctattgcgagcacgaccagaggcgcagagactgtggtgccctagggcatggagcttggaccctgcacagctgccgcctatgcaggtgcatcttctcagccctgtactgcctcccacaccaaacgttcagccactgtgacctgaaaagcttcctttcttcaggcgccagaggatcaagagaatgcagcatcccaagcctcctcctgctggtgctctgcctcctcctgcagggtgtggctggtaagggctgaggctcctaagtgcgatgatagactctcctcctgagctgtcacccttgattacaccacagtgtgccagcaagaaagctgggtggtgggcatctgacttggtgttgtgtcctgtaaataacagattcactggaatatgctggattctcatgctgtacaataaagaggcttaatggt

SEQ ID NO: 4 - protein, 202 aa, Mus musculus
MRANSPTQGISLKMHQARPLFLVTVALQLIGLGYSYQSEGDGAREVSNILSPVIPGTTLDRTLSNSSRKNDIPEGARLWDSLPDSSTLGESAVPVSRCCHNGGTCVLGSFCVCPAYFTGRYCEHDQRRRDCGALGHGAWTLHSCRLCRCIFSALYCLPHQTFSHCDLKSFLSSGARGSRECSIPSLLLLVLCLLLQGVAGKG

SEQ ID NO: 5 - DNA, 873 bp, Rattus norvegicus
  CDS (1)...(675): Encodes CFC1
atgcagatcccgaaggctagctatgcgcgcatccggaaccctagcccaggaagaccttggctaacgtccccagtgatgacgtatgtgaaaaagcaaaccctatgggcacgttgcccgtccacccagagtctcttcacatcagctgcacagctgcttatccatttgtggagcgggcagttgagagaaggagatggtgccagagaaattagctatctcctgtctccaaagctcccagggacgacactggatggaaccctgagtagttccagtagaaagaatgacagccaggagggagcgcacccgtcggaggccctttctggttccaacactttggaggagagtgcggtccctggatcccgctgttgccacaatggaggcacctgcgttctgggcagtttctgcgtgtgccccgcgcacttcacaggtcgctactgcgagcacgaccagagacacagagactgtggcgcactggggcacggagcttggaccctgcacagctgccgcctatgcaggtgcatcttctcagccctttactgcctcccacgccaaacattcagccactgtgacctgaaaagcttcctctcttcaggcgccagaggatcaagggcatgcagcatcccgagactcctcctgctggtgctctgcctcctcctgcaggttgtgtgtggctggtaagggctgaggctctgaactgccctgacagaatctcctcctgagcctgcaaggtgtcacccttgactacccatggcaccacagcacgcctgcaggaaagctgggtggtgggtatctgcttcagtgttgaatgctgtaaatgaccgaatggctgaaatatgctggatcctcgtgctgtgcaataaagacgttcaacagcga

SEQ ID NO: 6 - protein, 224 aa, Rattus norvegicus
MQIPKASYARIRNPSPGRPWLTSPVMTYVKKQTLWARCPSTQSLFTSAAQLLIHLWSGQLREGDGAREISYLLSPKLPGTTLDGTLSSSSRKNDSQEGAHPSEALSGSNTLEESAVPGSRCCHNGGTCVLGSFCVCPAHFTGRYCEHDQRHRDCGALGHGAWTLHSCRLCRCIFSALYCLPRQTFSHCDLKSFLSSGARGSRACSIPRLLLLVLCLLLQVVCGW